(19) United States
(12) Patent Application Publication
(10) Pub. No.: US 2008/0058300 A1
(43) Pub. Date: Mar. 6, 2008

(54) Induction of weight loss and the selective inhibition of PTP1B

(76) Inventors: Michael McLane, Plymouth Meeting, PA (US); Kristen A. Lantz, Plymouth Meeting, PA (US); Susan G. Emeigh Hart, Plymouth Meeting, PA (US); Andrew V. Albright, Plymouth Meeting, PA (US); Hsiao-Ling Hung, Plymouth Meeting, PA (US); Henry R. Wolfe, Plymouth Meeting, PA (US)

Correspondence Address: MORGAN LEWIS & BOCKIUS LLP, 1111 PENNSYLVANIA AVENUE NW, WASHINGTON, DC, 20004, US

(21) Appl. No.: 11/790,083

(22) Filed: Apr. 23, 2007

Related U.S. Application Data
(60) Provisional application No. 60/793,656, filed on Apr. 21, 2006.
(60) Provisional application No. 60/794,534, filed on Apr. 25, 2006.
(60) Provisional application No. 60/810,695, filed on Jun. 5, 2006.
(60) Provisional application No. 60/812,970, filed on Jun. 13, 2006.
(60) Provisional application No. 60/879,792, filed on Jan. 11, 2007.

(51) Int. Cl. A61K 31/575; A61P 3/00

(52) U.S. Cl. 514/182

(57) ABSTRACT

The present invention is directed to the use of compound 1436 for the induction of weight loss in an obese mammal.

### CROSS REFERENCE TO RELATED APPLICATIONS

This application claims priority to U.S. Provisional Application Nos. 60/793,656, filed Apr. 21, 2006; 60/794,534, filed Apr. 25, 2006; 60/810,695, filed Jun. 5, 2006; 60/812,970, filed Jun. 13, 2006; and 60/879,792, filed Jan. 11, 2007, each of which is incorporated by reference in its entirety.

### FIELD OF THE INVENTION

This application is directed to the use of compound 1436 for the induction of weight loss in an obese mammal.

### BACKGROUND OF THE INVENTION

Several aminosterol compounds have been isolated from the liver of the dogfish shark, Squalus acanthias. One of these compounds has been designated as 1436, the structure of which is shown in FIG. 1. Compound 1436 has been previously described in, e.g., U.S. Pat. Nos. 5,763,430; 5,795,885; 5,847,172; 5,840,936 and 6,143,738, each of which is incorporated in its entirety, and has been shown to inhibit weight gain and suppress appetite, which leads to weight loss, in animal models.

Obesity is a major medical problem in the United States and increasingly so in the rest of the developed world. The cause is primarily the effect of a sedentary life style and a fat-rich diet. The obese individual is susceptible to medical problems directly related to his obesity such as type II diabetes, insulin resistance, elevated serum cholesterol, high blood pressure, congenital obesity syndromes (including congenital leptin, pro-opiomelanocortin (POMC) and melanocortin-4 receptor (MC4R) deficiencies), and sleep apnea, especially in pickwickian syndrome. In addition, the accumulation of fat in the liver can progress to nonalcoholic steatohepatitis and cirrhosis. Another problem for obese individuals is an increased risk in any surgery that must cut through thick layers of fatty tissue that are highly vascularized and therefore prone to hemorrhage. Necessary surgery is frequently postponed until the obese patient can lose sufficient weight to make the risk of the operation acceptable.

Insulin is an important regulator of different metabolic processes and plays a key role in the control of blood glucose. Defects related to insulin synthesis and signaling lead to diabetes mellitus. Binding of insulin to the insulin receptor (IR) causes rapid autophosphorylation of several tyrosine residues in the intracellular part of the beta-subunit. Three closely positioned tyrosine residues (the tyrosine-1150 domain) must be phosphorylated to obtain maximum activity of the insulin receptor tyrosine kinase (IRTK), which transmits further signals via tyrosine phosphorylation of other cellular substrates, including insulin receptor substrate-1 (IRS-1) and insulin receptor substrate-2 (IRS-2).

Protein phosphorylation is a well-recognized cellular mechanism for transducing and regulating signals during different stages of cellular function (see, e.g., Hunter, Phil, Trans. R. Soc. Lond. B. 353: 583-605 (1998); Chan et al., Annu. Rev. Immunol. 12: 555-592 (1994); Zhang, Curr. Top. Cell. Reg. 35: 21-68 (1997); Matozaki and Kasuga, Cell. Signal. 8: 113-119 (1996)). There are at least two major recognized classes of phosphatases: (1) those that dephosphorylate proteins that contain a phosphate group(s) on a serine or threonine moiety (termed Ser/Thr phosphatases or duel specificity phosphatases or DSPs) and (2) those that remove a phosphate group(s) from the amino acid tyrosine (termed protein tyrosine phosphatases or PTPases or PTPs).

Several studies clearly indicate that the activity of the auto-phosphorylated IRTK can be reversed by dephosphorylation in vitro (reviewed in Goldstein, Receptor 3: 1-15 (1993)) with the tri-phosphorylated tyrosine-1150 domain being the most sensitive target for PTPases. This tri-phosphorylated tyrosine-1150 domain appears to function as a control switch of IRTK activity and the IRTK appears to be tightly regulated by PTP-mediated dephosphorylation in vivo (Faure et al., J. Biol. Chem. 267: 11215-11221 (1992)).

PTP1B has been identified as at least one of the major phosphatases involved in IRTK regulation through studies conducted both in vitro (Seely et al., Diabetes 45: 1379-1385 (1996)) and in vivo using PTP1B neutralizing antibodies (Ahmad et al., J. Biol. Chem. 270: 20503-20508 (1995)). Two independent studies have indicated that PTP1B knock-out mice have increased glucose tolerance, increased insulin sensitivity and decreased weight gain when on a high fat diet (Elchebly et al., Science 283: 1544-1548 (1999) and Klaman et al., Mol. Cell. Biol. 20: 5479-5489 (2000)). Overexpression or altered activity of tyrosine phosphatase PTP1B can contribute to the progression of various disorders, including insulin resistance and diabetes (Ann. Rev. Biochem. 54: 897-930 (1985)). Furthermore, there is evidence which suggests that inhibition of protein tyrosine phosphatase PTP1B is therapeutically beneficial for the treatment of disorders such as type I and II diabetes, obesity, autoimmune disorders, acute and chronic inflammation, osteoporosis and various forms of cancer (Zhang Z Y et al., Expert Opin. Investig. Drugs 2: 223-33 (2003); Taylor S D et al., Expert Opin. Investig. Drugs 3:199-214 (2004); J. Natl. Cancer Inst. 86: 372-378 (1994); Mol. Cell. Biol. 14: 6674-6682 (1994); The EMBO J. 12: 1937-1946 (1993); J. Biol. Chem. 269: 30659-30667 (1994); and Biochemical Pharmacology 54: 703-711(1997)).

The PTPase family of enzymes can be classified into two subgroups: (1) intracellular or nontransmembrane PTPases and (2) receptor-type or transmembrane PTPases. Most known intracellular type PTPases contain a single conserved catalytic phosphatase domain consisting of 220-240 amino acid residues. The regions outside the PTPase domains are believed to play important roles in localizing the intracellular PTPases subcellularly (Mauro, L. J. and Dixon J. E., TIBS 19: 151-155 (1994)). The first of the intracellular PTPases to be purified and characterized was PTP1B (Tonks et al., J. Biol. Chem. 263: 6722-6730 (1988)). Other examples of intracellular PTPases include (1) T-cell PTPase (TCPTP) (Cool et al., Proc. Natl. Acad. Sci. USA 86: 5257-5261 (1989)), (2) neuronal phosphatases STEP (Lombroso et al., Proc. Natl. Acad. Sci. USA 88: 7242-7246 (1991)), (3) PTP1C/SH-PTP1/SHP-1 (Plutzky et al., Proc. Natl. Acad. Sci. USA 89: 1123-1127 (1992)), (4) PTP1D/Syp/SH-PPT2/SHP-2 (Vogel et al., Science 259: 1611-1614 (1993); Feng et al., Science 259: 1607-1611(1993)).

Receptor-type PTPases consist of (a) a putative ligand-binding extracellular domain, (b) a transmembrane segment, and (c) an intracellular catalytic region. The structure and sizes of the putative ligand-binding extracellular domains of receptor-type PTPases are quite divergent. In contrast, the intracellular catalytic regions of receptor-type PTPases are very homologous to each other and to the intracellular PTPases. Most receptor-type PTPases have two tandemly duplicated catalytic PTPase domains. The first PTPase receptor subtypes identified were (1) CD45 (Ralph, S. J., EMBO J. 6: 1251-1257 (1987)) and (2) LAR (Streuli et al., J. Exp. Med. 168:1523-1530 (1988)). Since then, many more receptor subtypes have been isolated and characterized, including, e.g., PTPalpha, PTPbeta, PTPdelta, PTPepsilon and PTPxi. (Krueger et al. EMBO J. 9: 3241-3252 (1990)).

Although agents have been identified for use as PTP1B inhibitors, such as the heteroaryl- and aryl-amino acetic acids described in WO 01/19831, WO 01/19830, and WO 01/17516, these agents do not exhibit separation of the inhibitory activity between PTP1B and TCPTP. Furthermore, because of the potential immunosuppressive effects resulting from inhibiting TCPTP, selective inhibition of PTP1B over TCPTP would make such agents more suitable for drug development as they could diminish or eliminate undesired side effects resulting from such nonselectivity.

The dopamine and norepinephrine transporters, DAT and NET respectively, are located on pre-synaptic neurons of the hypothalamus and function to decrease the levels of synaptic dopamine or norepinephrine after it has been released into the synapse. When DAT or NET are inhibited, their levels rise in the synapse, activating their receptors.

Work by Billes and Cowley (Neuropsychopharmacology 1:1-13 (2006)), Gadde and Xiong (Expert Rev. Neurother., 7: 17-24 (2007)) and Gehlert et al. (J. Pharmacol. Exp. Ther., 287: 122-7 (1998)) has demonstrated that inhibitors of DAT and/or NET or agonists of their receptors can act as effective weight loss agents. Thus, the inhibition of DAT and NET observed in vitro in the present invention with compound 1436 supports this as part of a mechanism responsible for weight loss.

Therefore, there is a need for a drug that can safely induce rapid weight loss in obese individuals wherein the obesity is diet induced. A drug of this type would also be useful for the treatment of complications due to obesity, obesity in type II diabetes, high serum cholesterol, sleep apnea (especially in pickwickian syndrome), nonalcoholic steatohepatitis and surgery for obese patients.

### SUMMARY OF THE INVENTION

One aspect of the invention is a method for the rapid induction of weight loss in an exogenously obese subject by administration of compound 1436.

Another aspect of the invention is a method of treating, by administration of compound 1436, the complications associated with exogenous obesity including, but not limited to, type II diabetes, high serum cholesterol, the incidence of heart attack and stroke, sleep apnea (especially in pickwickian syndrome), congenital obesity syndromes (including congenital leptin, POMC and MC4R deficiencies), nonalcoholic steatohepatitis and complications in surgery due to obesity.

A further aspect is the treatment of an exogenously obese mammal with compound 1436 to produce a significant reduction of percent body fat

In another aspect of the invention, a PTP1B inhibitor in the form of compound 1436, which demonstrates selective inhibitory activity for PTP1B over other phosphatases, is provided.

In an exemplary embodiment, the present invention is directed to the compound of FIG. 1 (i.e., compound 1436) or a therapeutically acceptable salt or prodrug thereof.

Another aspect of the invention is a pharmaceutical composition comprising a therapeutically effective amount of compound 1436 in combination with a pharmaceutically acceptable carrier.

Another aspect of the invention relates to a method of selectively inhibiting protein tyro sine phosphatase 1B over T-c ell protein tyro sine phosphatase comprising administering a therapeutically effective amount of compound 1436.

Another aspect of the invention relates to a method of treating disorders caused by overexpressed protein tyrosine phosphatase 1B or enhanced activity of protein tyrosine phosphatase 1B, comprising administering a therapeutically effective amount of compound 1436 to a recipient in need thereof.

Another aspect of the invention relates to a method of treating type I and type II diabetes mellitus, comprising administering a therapeutically effective amount of compound 1436 to a recipient afflicted with either of these diseases.

Another aspect of the invention relates to a method of treating obesity, comprising administering a therapeutically effective amount of compound 1436 to a recipient who is suffering from obesity.

Another aspect of the invention relates to a method of treating disorders by increasing the amounts of dopamine or norepinephrine in the vicinity of their reuptake transporters, comprising administering a therapeutically effective amount of compound 1436 to a recipient in need thereof.

### BRIEF DESCRIPTION OF THE FIGURES

FIGS. 1-12 below are only illustrative embodiments of the scope of the present invention and are not intended to otherwise limit the scope of the invention.

FIG. 1 shows the structure of compound 1436.

FIG. 2 shows the effect of a high fat diet on murine body weight and the reversal by treatment with compound 1436.

FIG. 3 shows the % Change in Body Weight for mice on the 60% fat diet with various treatments.

FIG. 4 shows the % Change in Body Weight for mice on the 45% fat diet with various treatments.

FIG. 5 shows the % Change in Body Weight for mice on the 10% fat diet with various treatments.

FIG. 6 shows the Body Composition of mice on a 60% fat diet with various treatments.

FIG. 7 shows the Body Composition of mice on a 45% fat diet with various treatments.

FIG. 8 shows the Body Composition of mice on a 10% fat diet with various treatments.

FIG. 9 shows the histology of brown adipose tissue in normal mice and mice on a 60% fat diet with various treatments.

FIG. 10 shows the dose-response curves for the inhibition of cellular uptake of dopamine and norepinephrine by MSI-1436.

FIG. 11 shows western blot analyses of the effect of MSI-1436 on the extent of phosphorylation of insulin receptor A.

FIG. 12 shows western blot analyses of the effect of MSI-1436 on the extent of phosphorylation of insulin receptor substrate-1.

FIG. 13 shows the histology of the white adipose tissue in normal mice and mice on a 60% fat diet with various treatments.

### DETAILED DESCRIPTION OF THE INVENTION

### Definitions

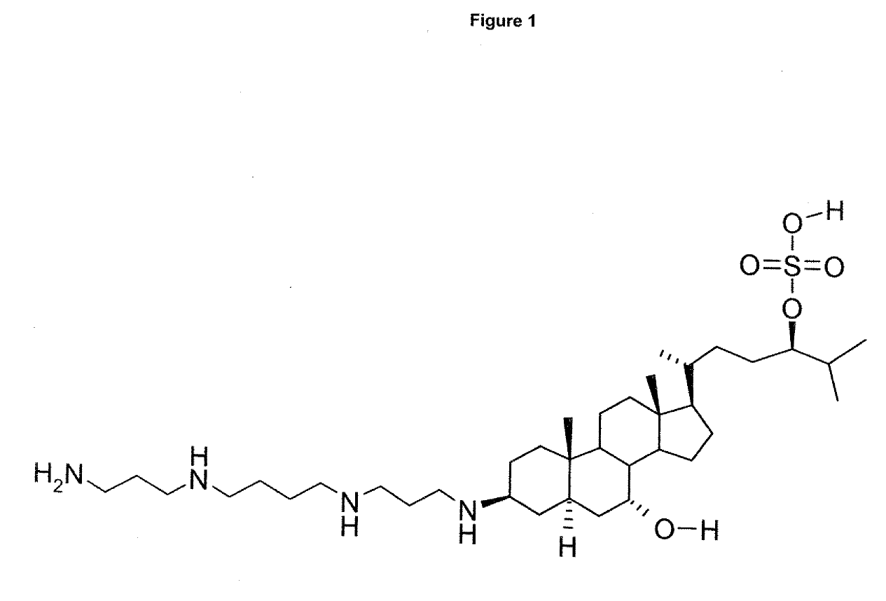

As used herein, “compound 1436” or “MSI-1436” or simply “1436” refers to the aminosterol represented structurally in FIG. 1. Compound 1436 is also intended to encompass pharmaceutically acceptable salts of the free base compound.

As used herein, the term “obese” as it pertains to humans includes, but is not limited to, a human with a Body Mass Index Score greater than at least about 30.

As used herein, the term “obese” as it pertains to mice includes, but is not limited to a mouse with a % total body fat greater than at least about 25%.

As used herein, the term “exogenously obese” refers to obesity due to overeating.

As used herein, the term “pickwickian syndrome” refers to a complex of obesity, somnolence, hypoventilation, and erythrocytosis.

As used herein, the term “nonalcoholic steatohepatitis” refers to an inflammatory disease of the liver most frequently found in obese women with type II diabetes.

As used herein, the term “osteoarthritis” refers to a noninflammatory degenerative joint disease that is aggravated by obesity.

As used herein, the term “basal metabolic rate” or BMR refers to the number of calories a mammal, including a human, burns at rest to maintain normal body functions.

As used herein, the term “reduced caloric intake” refers to a decrease in the amount of calories consumed by a mammal including, but not limited to, a human.

As used herein, the phrase “selective inhibition of protein tyrosine phosphatase-1B (PTP1B) over T-Cell protein tyrosine phosphtase-1B (TCPTP)” refers to the inhibition of protein tyrosine phosphatase-1B with an IC₅₀ value that is at least about 40 fold less than the IC₅₀ value for T-Cell protein tyrosine phosphtase-1B. In particular embodiments, the IC₅₀ value for the inhibition of PTP1B is at least about 50 fold less or at least about 60 fold less or at least about 70 fold less or at least about 80 fold less or at least about 90 fold less or at least about 100 fold less or at least about 200 fold less than the IC₅₀ value for inhibition of TCPTP.

As used herein, the term “inhibitor” refers to a compound which prevents the binding of PTP1B to its endogenous substrates and/or prevents the dephosphorylation mediated by PTP1B on its endogenous substrate, including but not limited to, insulin receptor tyrosine kinase (IRTK), and the fragments of IRTK, and the unnatural substrates, such as, for example, p-nitrophenyl phosphate.

As used herein, the term “selective” refers to a compound having at least about 3-fold greater inhibition in terms of a IC₅₀ value for the PTP1B enzyme compared with the IC₅₀ value of other enzymes, including but not limited to, TC-PTP, SHP-2, LAR, CD45, PP2B and Cdc25c.

As used herein, the “effective amount” is an amount sufficient to effect beneficial or desired results. For example, a therapeutic amount is one that achieves the desired therapeutic effect. This amount may be the same or different from a prophylactically effective amount, which is an amount necessary to prevent the onset of disease or disease symptoms. An effective amount can be administered in one or more administrations, applications or dosages. In one embodiment, an effective amount is an amount of compound 1436 which induces weight loss in an obese individual.

Methods of Treatment

The aminosterol compound 1436 has been shown in the present invention to induce rapid weight loss in diet induced obese mice. In addition, the observed weight loss is greatest in the most obese mice and is due predominately to the loss of fat but little if any loss of protein. Also, the most obese 1436-treated mice were observed to lose significantly more weight than their pair-fed counter parts, indicating an induction of weight loss that is caused by more than appetite suppression. Surprisingly, it was observed that after a period of treatment, such as for example about 7 to about 10 days of treatment, the rate of weight loss appeared to decline for the pair-fed mice whereas the decline in rate was less for the 1436-treated mice. The difference in final weight between the 1436-treated and pair-fed groups appears to be at least partially due to a relatively greater loss of fat by the 1436-treated group.

The most common method for treating exogenous obesity is diet restriction. This method has three major problems: (1) individual compliance; (2) the tendency to lose significant amounts of muscle mass as well as fat; and (3) the resetting of the individual's metabolism to a lower level.

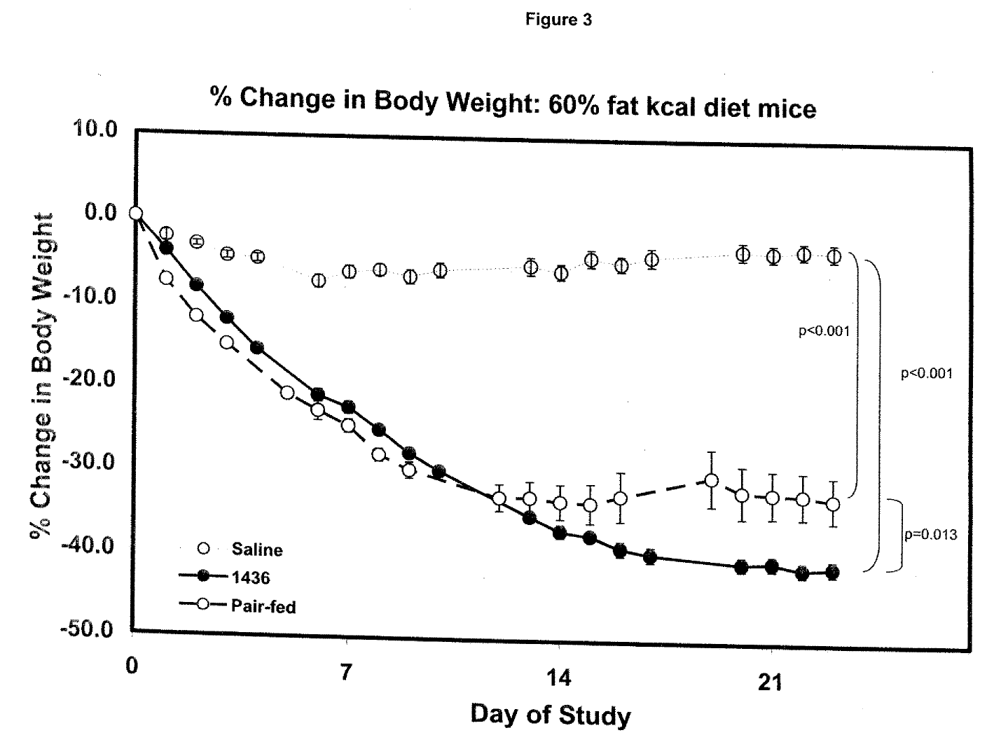

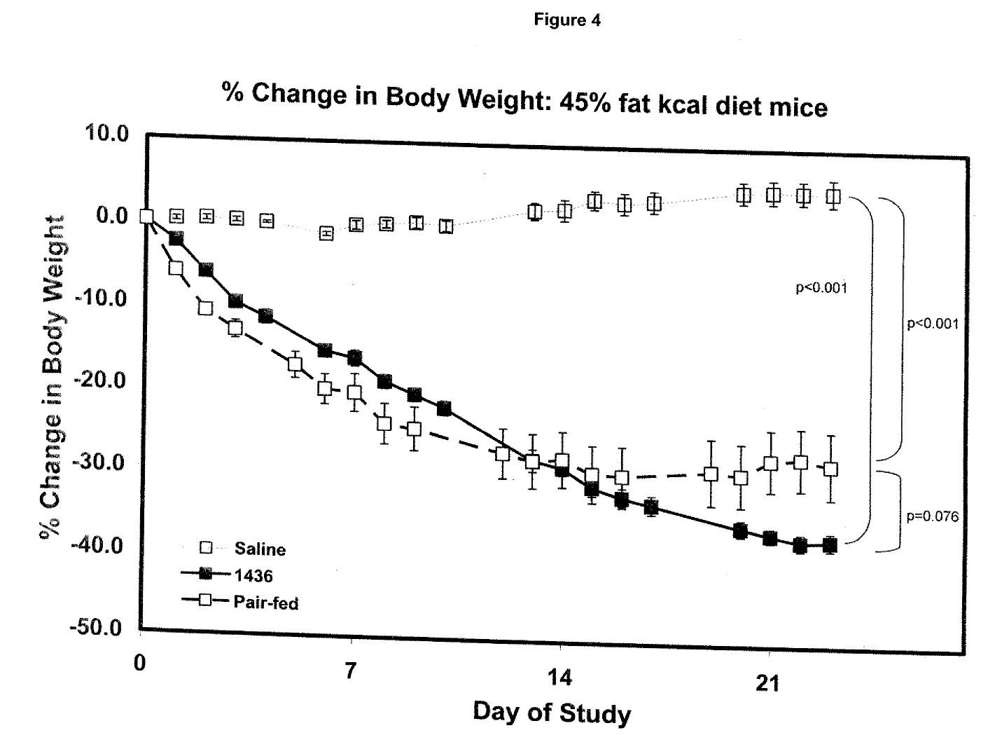

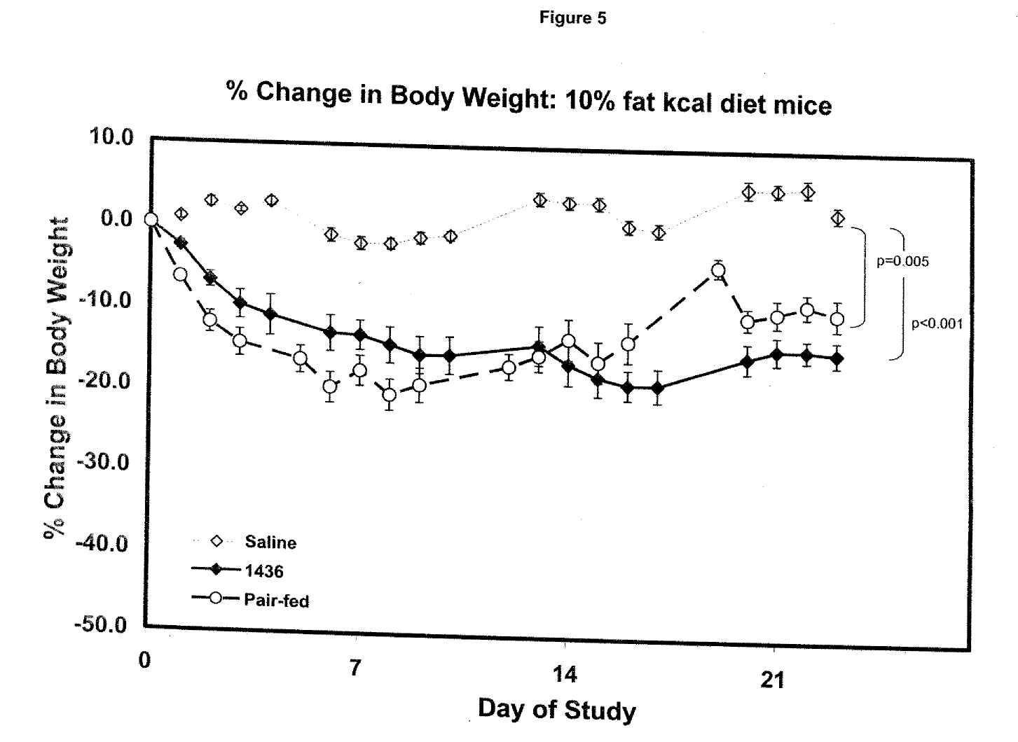

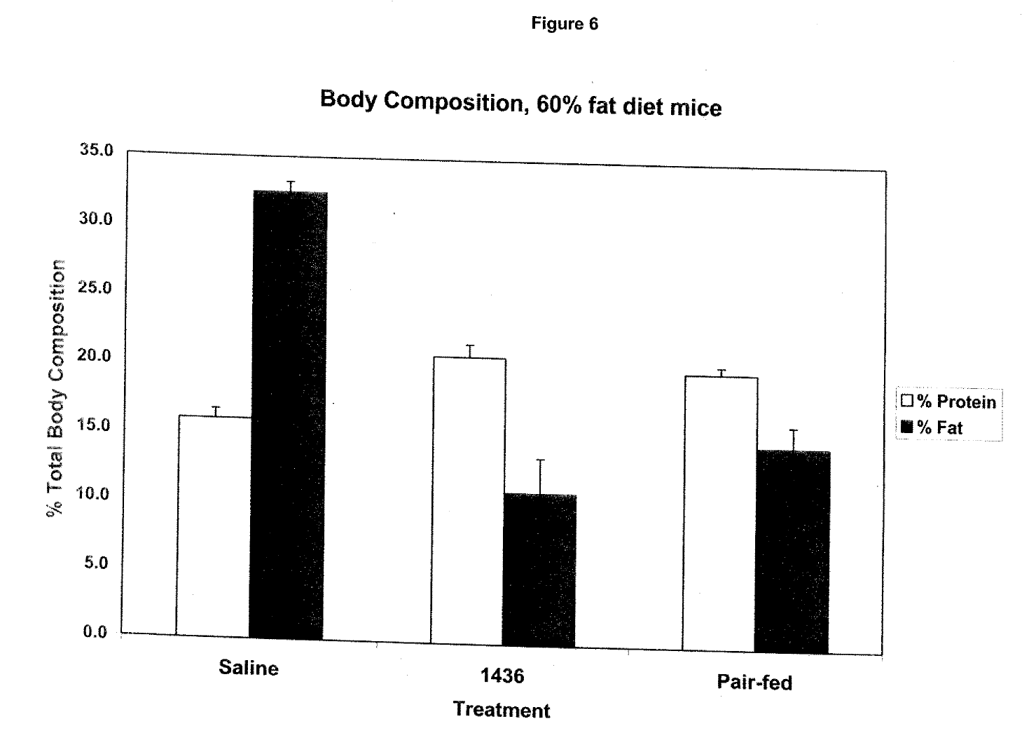

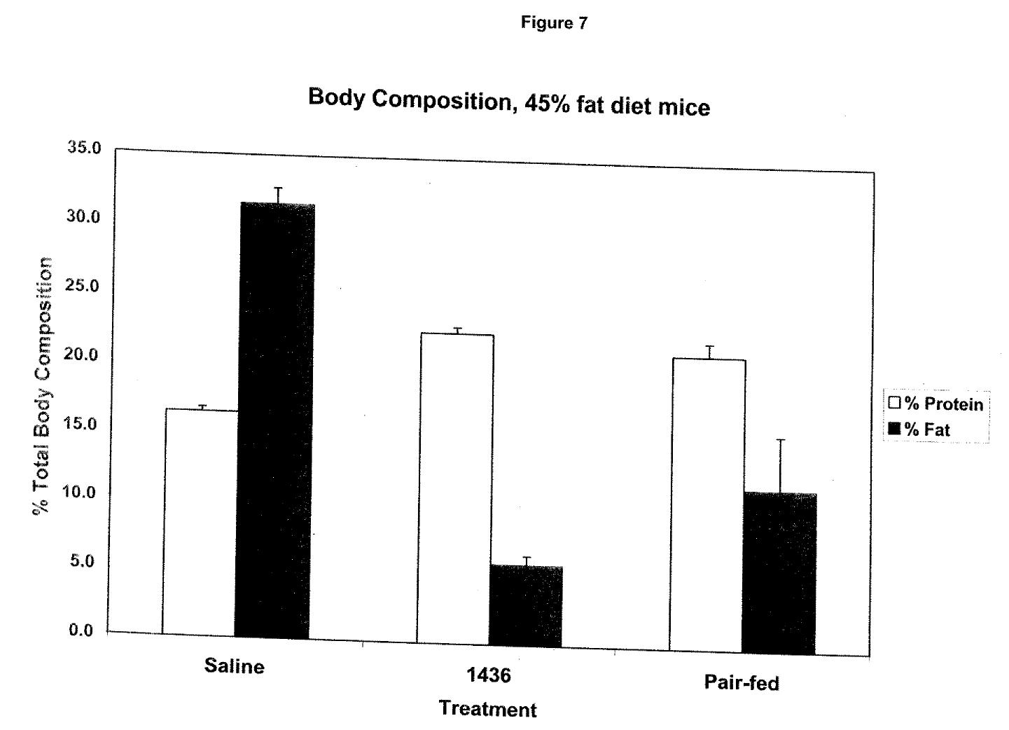

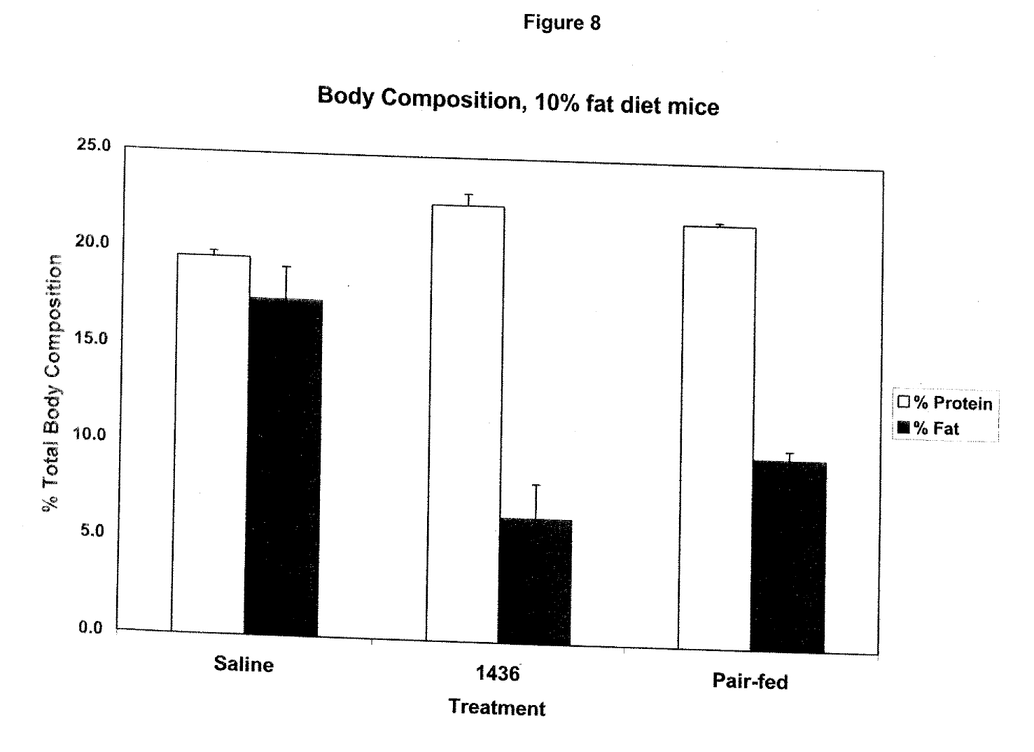

Treatment with compound 1436 overcomes all of these problems. Physician controlled drug treatment will greatly improve individual compliance. As shown in FIGS. 6-9 and 13 there is a significant loss of fat after treatment with compound 1436 with little concomitant loss of protein. As shown in FIGS. 3-5 the resetting of the metabolism does not seem to occur as the 1436-treated animals continue to lose weight even when the weight loss for the diet-treated animals has leveled off.

Pharmaceutically-acceptable salts of compound 1436 include the conventional non-toxic salts or the quaternary ammonium salts which are formed from inorganic or organic acids or bases. Examples of such acid addition salts include acetate, adipate, benzoate, benzenesulfonate, citrate, camphorate, dodecylsulfate, hydrochloride, hydrobromide, lactate, maleate, methanesulfonate, nitrate, oxalate, pivalate, propionate, succinate, sulfate and tartrate. Base salts include ammonium salts, alkali metal salts such as sodium and potassium salts, alkaline earth metal salts such as calcium and magnesium salts, salts with organic bases such as dicyclohexylamine salts and salts with amino acids such as arginine. Also, the basic nitrogen-containing groups may be quaternized with, for example, alkyl halides.

In addition to carriers, the pharmaceutical compositions of the invention may also include stabilizers and preservatives. For examples of typical carriers, stabilizers and adjuvants known to those of skill in the art, see Remington: The Science and Practice of Pharmacy, 21st ed. (Lippincott, Williams & Wilkins, PA (2005)).

Compound 1436 may be administered alone or preferably as a pharmaceutical formulation comprising 1436 together with at least one pharmaceutically acceptable carrier. Optionally, other therapies known to those of skill in the art may be combined with the administration of the 1436.

In vivo administration of compound 1436 can be effected in one dose, multiple doses, continuously or intermittently throughout the course of treatment. Doses range from about 0.05 mg/kg to about 5 mg/kg, such as between about 0.07 mg/kg to about 3 mg/kg, such as between about 0.1 mg/kg to about 2 mg/kg, such as between about 0.3 mg/kg to about 1.5 mg/kg, such as between about 0.5 mg/kg to about 1 mg/kg, in single or divided daily doses. Methods of determining the most effective means and dosage of administration are well known to those of skill in the art and will vary with the composition used for therapy, the purpose of the therapy, the target cell being treated and the subject being treated. Single or multiple administrations can be carried out with the dose level and pattern being selected by the treating physician. Extended, delayed and/or sustained release formulations of compound 1436 can also be administered.

Pharmaceutical compositions containing compound 1436 can be administered by any suitable route, including oral, rectal, intranasal, topical (including transdermal, aerosol, buccal and sublingual), parenteral (including subcutaneous, intramuscular, intravenous), intraperitoneal and pulmonary. It will be appreciated that the preferred route will vary with the condition and age of the recipient, and the disease being treated.

### EXAMPLES

### Example 1

### Induction of Weight Loss in Diet Induced Obese Mice

Mice and Study Design. At weaning, male AKR/J mice from Jackson Laboratories (Bar Harbor, Me., USA) were placed on a 10%, 45%, or 60% fat kcal diet (Research Diets, Inc., New Brunswick, N.J., USA). Mice were weighed weekly and treatment began after 13-14 weeks of access to the different fat diets. Immediately before the start of treatment, mice from all three diets were randomly subdivided further into three groups within the same fat composition diet with an even weight distribution. All mice were dosed (i.p. route) on a q7dx4 schedule, where the first dose of 1436 was 10 mg/kg and all remaining doses were 5 mg/kg. Saline-treated animals were administered 10 mL/kg. Pair-fed animals were dosed with saline (10 mL/kg) on the same q7dx4 schedule on a 1-day stagger from the other groups. As a measure of food consumption, remaining food was weighed daily. Pair-fed groups were allotted the exact amount of food consumed by 1436-treated groups in the 24 hours prior. All procedures involving mice were performed in accordance with protocols approved by the Institutional Animal Care and Use Committee.

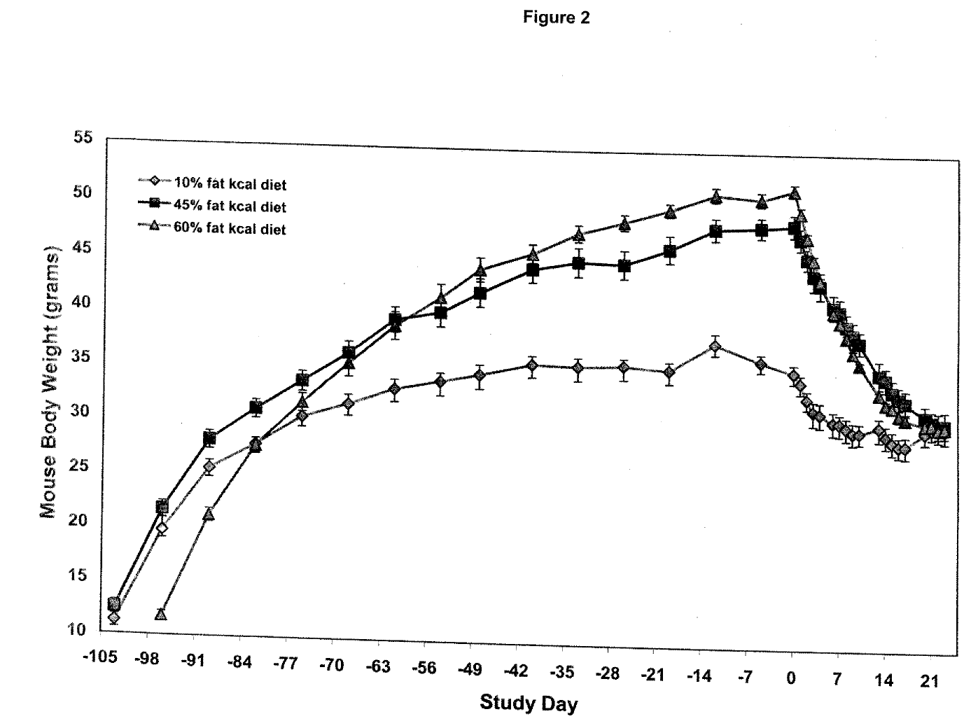

As seen in FIG. 2 the mice on the 45% and 60% fat diets became obese while those on the 10% fat diet (the normal mouse diet) gained weight normally. The animals on the 60% fat diet were significantly heavier than animals fed the 45% diet and both had significantly heavier than those fed the 10% fat diet. When the three groups were treated with 1436 (initiated Day 0 in FIG. 2) all three groups lost weight but the more obese the mouse the more rapid the weight loss and the greater the weight loss as all three groups arrived at about the same weight at the end of three weeks (FIG. 2).

FIGS. 3-5 show that the effect of 1436 on weight loss is greater than the effect of diet alone (based, e.g., on a comparison of the weight loss in the 1436-treated group with the weight loss in the pair-fed group). This effect was greatest in the 60% fat group where the p value was 0.013. This observation supports the conclusion that compound 1436 also induces weight loss by preventing the resetting of the basal metabolic rate that is normally seen in diet-induced weight loss.

Body Composition. Body composition (percent moisture, fat, protein and ash) was determined according to methods described with modifications (Official Methods of Analysis of AOAC INTERNATIONAL, 2000). Briefly, samples were dried at 125° C. for 4 hours to determine moisture. After drying, pentane was dripped through the sample for 5 hours to determine fat composition. Protein was determined through the combustion method for nitrogen detection and a factor of 6.25 was used to convert percent nitrogen to percent protein. To determine ash, samples were ignited at 550° C. for 5 hours and quantified gravimetrically.

FIGS. 6-8 show the total body composition at the end of the study for the 10%, 45% and 60% fat diet groups. Pre-treatment diet effects show a % body fat of greater than 30% for the high fat diets but below 20% for the normal diet. The 1436-treated and pair-fed groups both lose significant % body fat on all diets. There is a trend for greater loss of body fat in the 1436-treated group as compared to the pair-fed groups.

Histology. Intrascapular brown adipose tissue was fixed in 10% zinc formalin (Fisher Scientific, Kalamazoo, Mich., USA) for 48 hours and transferred to 70% EtOH. Tissues were embedded in paraffin and 5 μM sections were applied to glass microscope slides. Sections were stained with hematoxylin/eosin and images were captured with an Insight 18.2 Color Mosaic Camera (Diagnostic Instruments, Sterling Heights, Mich., USA) on an Olympus AX70 microscope (Melville, N.Y., USA).

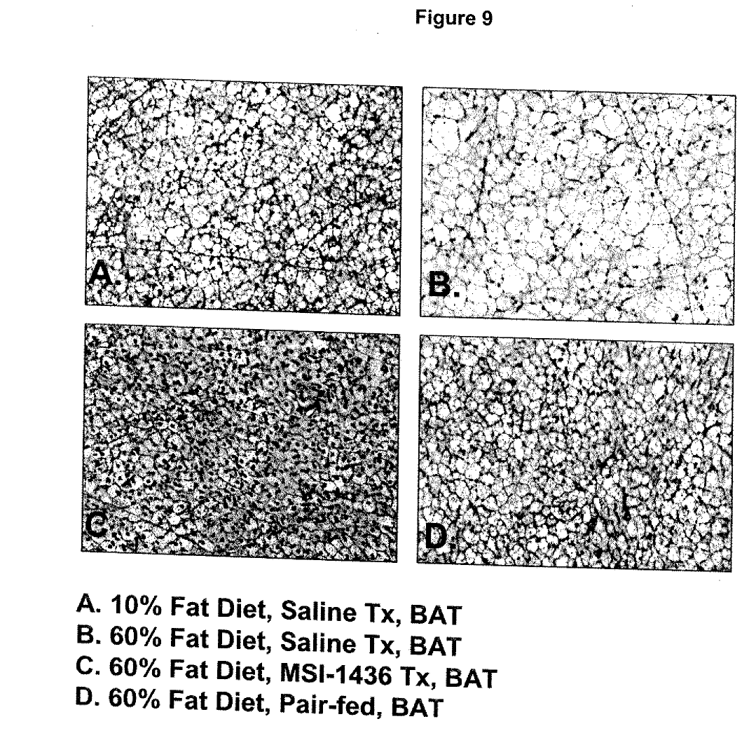

FIG. 9 shows the amount of fat (white globules) in brown adipose tissue (BAT) in a normal mouse (10% fat diet treated with saline, panel A), a mouse on the 60% diet treated with saline (panel B), a mouse on the 60% diet treated with 1436 (panel C) and the pair-fed mouse on a 60% diet. The 1436-treated mouse has lost all of the fat gained by the 60% diet and more than the mouse on restricted diet alone (pair-fed).

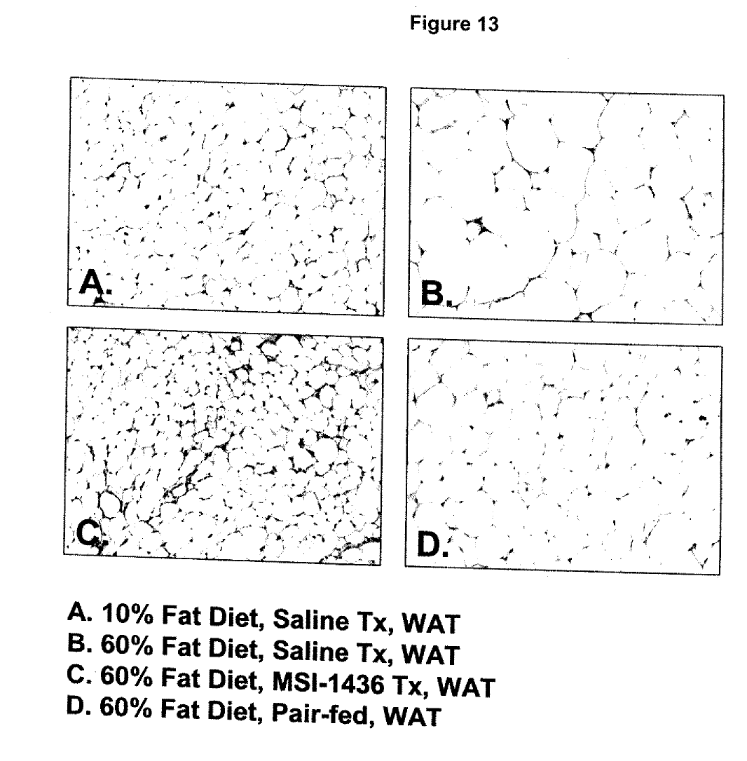

FIG. 13 shows the amount of fat (white globules) in white adipose tissue (WAT) in a normal mouse (10% fat diet treated with saline, panel A), a mouse on the 60% diet treated with saline (panel B), a mouse on the 60% diet treated with compound 1436 (panel C) and the pair-fed mouse on a 60% diet. The 1436-treated mouse has lost all of the fat gained by the 60% diet and more than the mouse on restricted diet alone (pair-fed).

### Example 2

### Selective inhibition of Tyrosine Phosphatase Enzymes

Inhibition of PTP1B and TCPTP by compound 1436 was determined under contract by MDS Pharma in enzyme assays using human recombinant proteins expressed in E. Coli and tyrosine phosphopeptide (20 μg/mL) or DiFMUP (10 μM) as the substrates, respectively. In the PTP1B assay, phosphatase activity was quantitated through ELISA analysis of remaining tyrosine phosphopeptide after 30-minute incubations at room temperature with various concentrations of 1436 (0.05 μM to 500 EM). The IC₅₀ value (1.14 μM) was determined using Data Analysis Toolbox™ (MDL Information Systems) by a non-linear, least squares regression analysis (See Table 1). The TCPTP assay required a 15-minute preincubation of enzyme and substrate at 37° C. followed by 60-minute incubations at 37° C. with various concentrations of 1436 (0.5 μM to 50 μM). TCPTP activity was assessed by spectrofluorimetric quantitation of DiFMU. Since no inhibition was demonstrated at any of the concentrations of 1436 tested, the IC₅₀ was estimated at >50 μM (Table 1).

TABLE 1
IC₅₀ Values for 1436 Inhibition of Tyrosine Phosphatase Enzymes

| | PTP1B | TCPTP |
|---|---|---|
| ˜IC₅₀ (μM) | 1.14 | >50.0 |

### Example 3

### Inhibition of Binding to the Dopamine (DAT) and Norepinephrine Transporters (NET)

Inhibition of binding to the Dopamine Transporter (DAT) and Norepinephrine Transporter (NET) by MSI-1436 was determined in radioligand binding assays using human recombinant proteins expressed in CHO-K1 or MDCK cells, respectively. Membrane preparations were incubated with [¹²⁵I] RTI-55 (0.15 nM for DAT, 0.20 nM for NET) in the presence of 1436 (0.005 μM to 50 μM) for 3 hours at 4° C. IC₅₀ values were determined as in PTP1B assays (See Table 2).

TABLE 2
IC₅₀ Values for 1436 Inhibition of Binding to DAT and NET

| | DAT | NET |
|---|---|---|
| ˜IC₅₀ (μM) | 1.74 | 3.24 |

### Example 4

### Inhibition of Cellular Uptake of Dopamine and Norepinephrine by MSI-1436

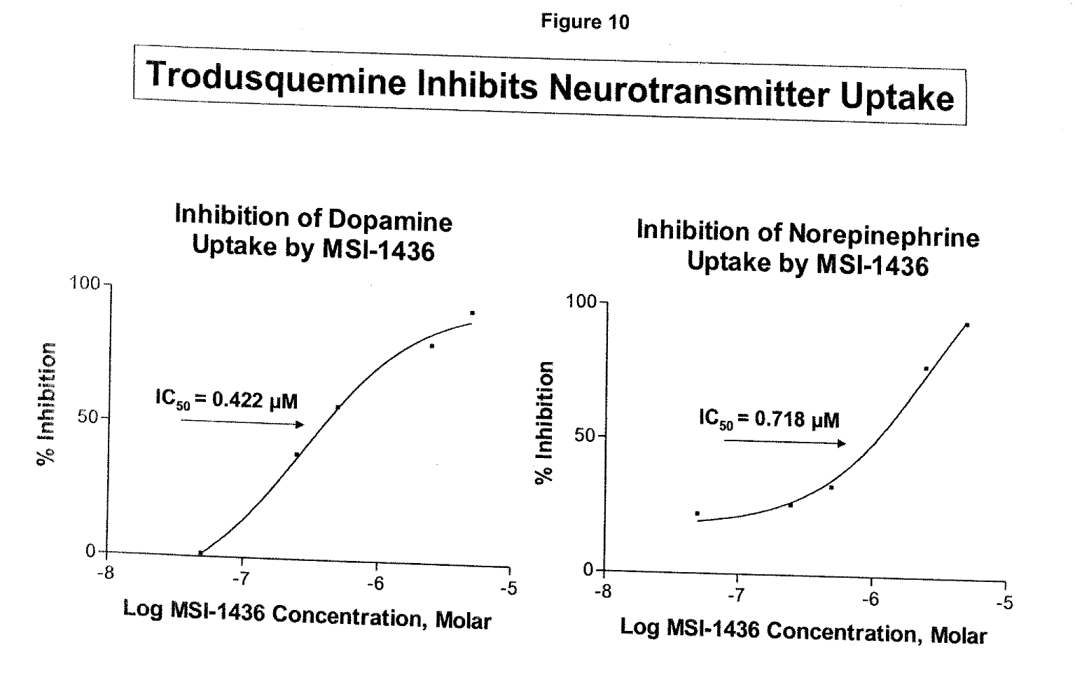

Dopamine and norepinephrine uptake in the presence of compound 1436 (0.05 μM to 5 μM) was investigated using CHO-K1 cells expressing dopamine transporters and MDCK cells expressing norepinephrine transporters. After 10-minute incubations of the cells with radioactive ligands in the presence of compound at room temperature, quantitation of [³H] Dopamine or [³H] Norepinephrine revealed an antagonistic action of 1436. Significance criteria for antagonism was met if there was ≧50% inhibition of uptake as compared to the induced response of nomifensine (DAT) or desipramine (NET). Table 3 details the antagonism measured and FIG. 10 shows the dose-response curves. The IC₅₀ values were calculated to be 422 nM for inhibition of Dopamine uptake and 718 nM for the inhibition of Norepinephrine uptake.

TABLE 3
Inhibition of Uptake of Dopamine and Norepinephrine by 1436

1436

| | Concentration (μM) | % Inhibition |
|---|---|---|
| Dopamine Uptake | 5.0 | 94 |
| | 2.5 | 81 |
| | 0.5 | 57 |
| | 0.25 | 39 |
| | 0.05 | 1 |
| Norepinephrine Uptake | 5.0 | 95 |
| | 2.5 | 78 |
| | 0.5 | 33 |
| | 0.25 | 26 |
| | 0.05 | 22 |

### Example 5

### In Vitro and In Vivo Inhibition of Protein Tyrosine Phosphatase 1B

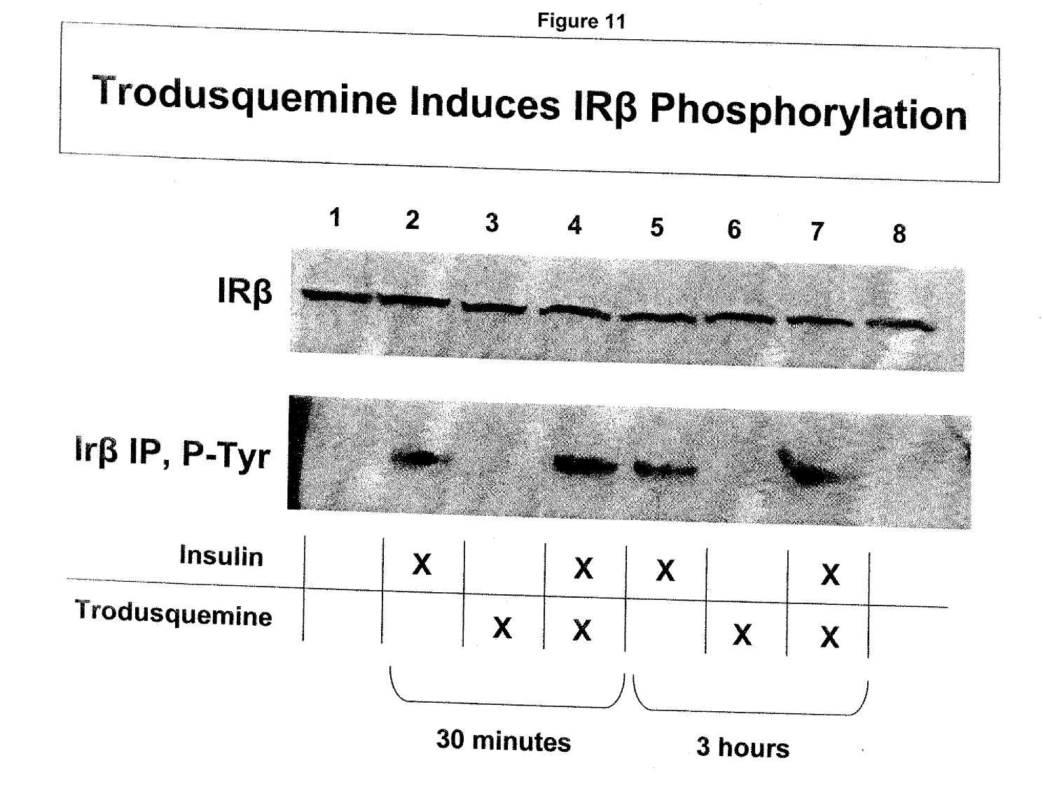

HepG2 cells (immortalized hepatocyte cells) were treated with 10 nM insulin, 10 jMMSI-1436, neither, or both for either 30 minutes or 3 hours in vitro. FIG. 11 shows Western blot analyses of cell lysates. No difference in the amounts of insulin receptor-beta (IR-beta) was noted (Panel A). Immunoprecipitation of insulin receptor beta, followed by phosphorylated tyrosine Western blot analyses, showed that insulin alone induced phosphorylation of IR-beta and MSI-1436 did not induce phosphorylation (Panel B). Cells treated with both insulin and MSI-1436 for 30 minutes demonstrated increased IR-beta phosphorylation as compared to insulin-induced phosphorylation (Panel B). This effect was slightly diminished at 3 hours.

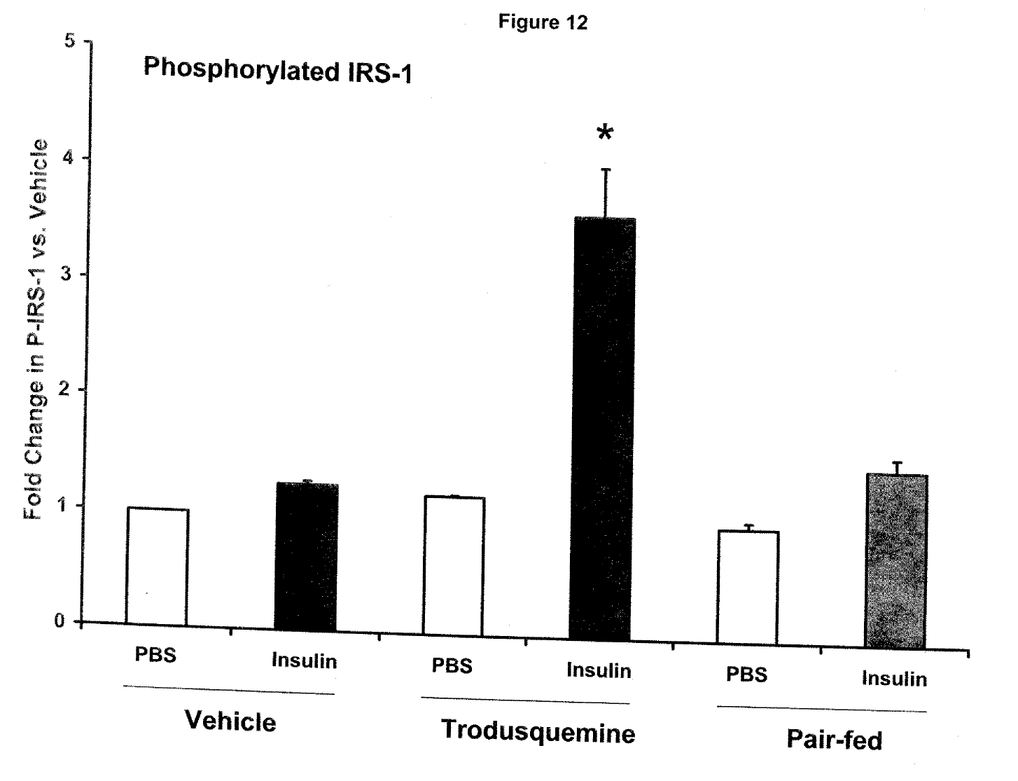

To examine the effect of MSI-1436 on PTP1B ex vivo, ob/ob mice were treated with either vehicle, MSI-1436 (single dose, 10 mg/kg, i.p.) or pair-fed control mice (n=4 per group). Twenty-four hours after the single treatment, mice were anesthetized and portal vein was exposed via an incision. Insulin or phosphate buffered saline (PBS) was injected into the portal vein and livers were harvested 2 min after the injection. Livers were lysed and lysates were immunoprecipitated with anti-insulin receptor substrate-1 (IRS-1) and then blotted for phosphotyrosine with the resultant blots representing phosphorylated insulin receptor substrate-1 (P-IRS-1). The blots were analyzed using imaging software. FIG. 12 showed that MSI-1436 in vivo enhanced the insulin-induced phosphorylation of IRS-1 and did so to a greater degree than pair-fed effect on insulin induction (FIG. 12).

Unless defined otherwise, all technical and scientific terms herein have the same meaning as commonly understood by one of ordinary skill in the art to which this invention belongs. Although methods and materials similar or equivalent to those described herein can be used in the practice or testing of the present invention, the preferred methods and materials are described herein. All patents and publications cited herein are incorporated herein by reference in their entirety.

We claim:

1. A method for inducing weight loss in an obese mammal, comprising: administering an effective amount of a composition comprising a pharmaceutically acceptable carrier or excipient and an aminosterol compound according to the following formula: 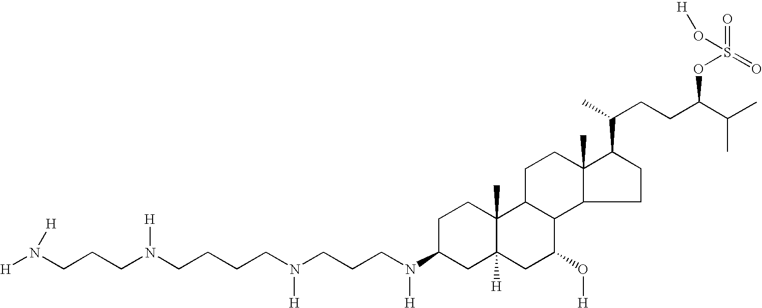
or a pharmaceutically acceptable salt thereof.

2. The method of claim 1 wherein the mammal also suffers from a condition selected from the group consisting of: type II diabetes, high serum cholesterol, sleep apnea, pickwickian syndrome and nonalcoholic steatohepatitis.

3. The method of claim 1 wherein the mammal is exogenously obese.

4. A method of claim 2 wherein the condition is type II diabetes.

5. A method of claim 2 wherein the condition is high serum cholesterol.

6. A method of claim 2 wherein the condition is sleep apnea.

7. A method of claim 2 wherein the condition is pickwickian syndrome.

8. A method of claim 2 wherein the condition is nonalcoholic steatohepatitis.

9. The method of claim 1 wherein the obese mammal is in need of a surgical procedure.

10. A method for inducing rapid loss of body fat in an exogenously obese mammal, comprising the step of: administering an effective amount of a composition comprising a pharmaceutically acceptable carrier or excipient and an aminosterol compound according to the following formula: 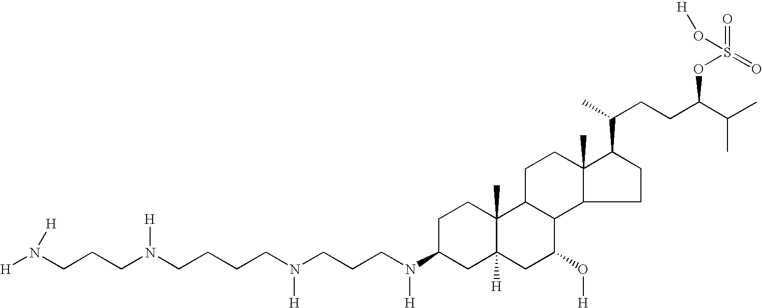
or a pharmaceutically acceptable salt thereof.

11. A method for sensitizing insulin in a mammal, comprising: administering to a mammal in need thereof an effective amount of an aminosterol compound according to the following formula: 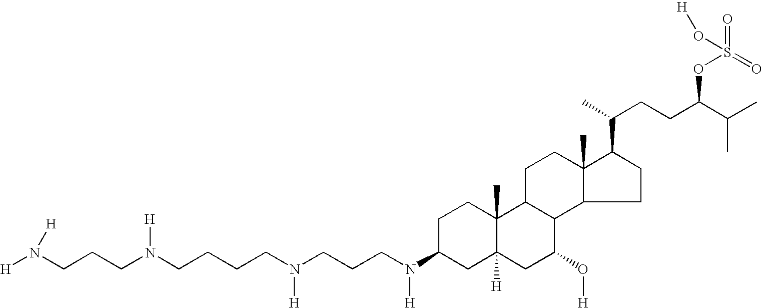
or a pharmaceutically acceptable salt thereof.

12. A method according to claim 11 wherein the mammal is a human.

13. A method according to claim 11 wherein the sensitization of insulin comprises a treatment of type I or type II diabetes.

14. A method according to claim 11 wherein the sensitization of insulin comprises a treatment of obesity.

15. A method of selectively inhibiting the enzyme PTP1B over the enzyme TCPTP in a mammal, comprising: administering to a mammal in need thereof an effective amount of an aminosterol compound according to the following formula: 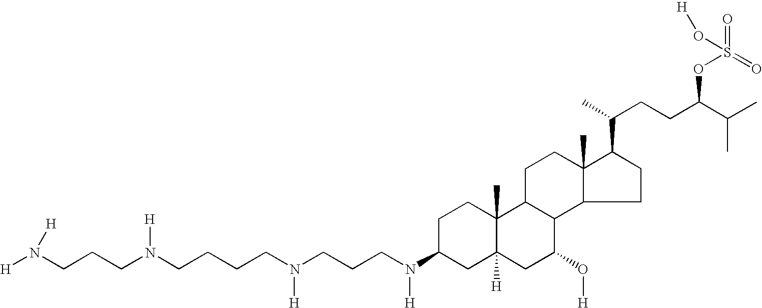
or a pharmaceutically acceptable salt thereof.

16. A method according to claim 15 wherein the enzymes PTP1B and TCPTP are human.

17. A method according to claim 15 wherein the mammal is a human.

18. A method according to claim 15 wherein the selective inhibition of the enzyme PTP1B over the enzyme TCPTP comprises an improved treatment for type I or type II diabetes.

19. A method according to claim 15 wherein the selective inhibition of the enzyme PTP1B over the enzyme TCPTP comprises an improved treatment for obesity.

20. The method according to claim 11 or claim 15, wherein the compound further comprises a pharmaceutically acceptable carrier or excipient.

21. The method according to claim 11 or claim 15, wherein the compound is administered via subcutaneous injection.

22. The method according to claim 11 or claim 15, wherein the compound is administered via inhalation.

23. A pharmaceutical composition for the sensitization of insulin in a mammal comprising a compound of the following formula:

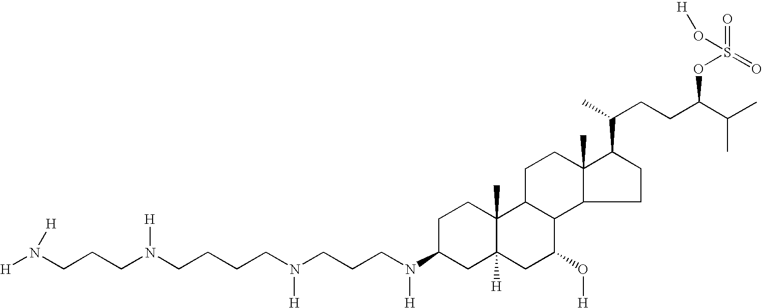

24. A method of maintaining the basal metabolic rate in a mammal with a reduced caloric intake comprising administering to the mammal an amount of a composition comprising a pharmaceutically acceptable carrier or excipient and an aminosterol compound according to the following formula: 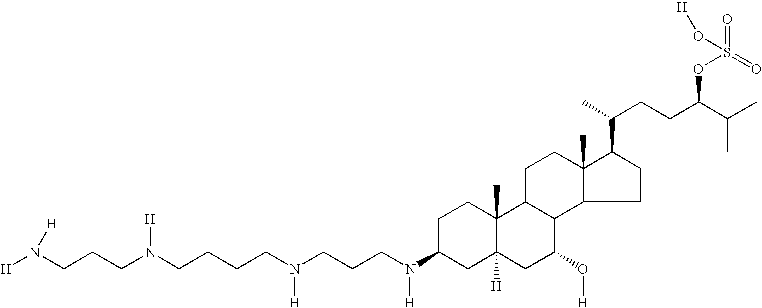
wherein the aminosterol compound is administered in an amount effective to maintain the metabolic rate of said mammal.

25. The method of any of claims 11 to 24 wherein the mammal is a human.

26. A method of inhibiting the dopamine uptake in a mammal, comprising: administering to a mammal in need thereof an effective amount of an aminosterol compound according to the following formula: 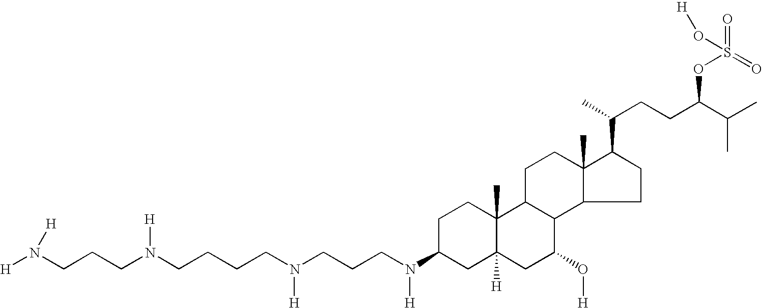
or a pharmaceutically acceptable salt thereof.

27. A method according to claim 26 wherein the mammal is a human.

28. A method according to claim 26 wherein the inhibition of dopamine uptake comprises an improved treatment for type I or type II diabetes.

29. A method according to claim 26 wherein the inhibition of dopamine uptake comprises an improved treatment for obesity.

30. A method of inhibiting the norepinephrine uptake in a mammal, comprising: administering to a mammal in need thereof an effective amount of an aminosterol compound according to the following formula: 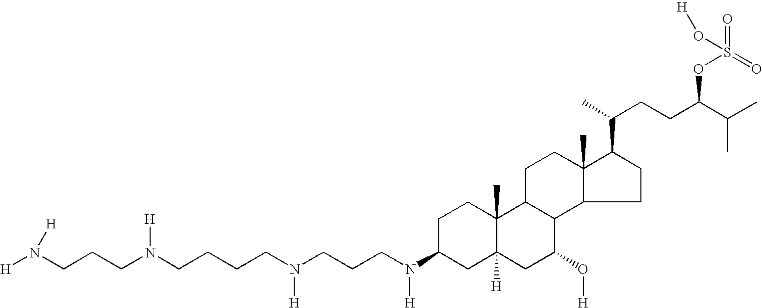
or a pharmaceutically acceptable salt thereof.

31. A method according to claim 30 wherein the mammal is a human.

32. A method according to claim 30 wherein the inhibition of norepinephrine uptake comprises an improved treatment for type I or type II diabetes.

33. A method according to claim 30 wherein the inhibition of norepinephrine uptake comprises an improved treatment for obesity.

34. The method according to claim 26 or claim 30, wherein the compound further comprises a pharmaceutically acceptable carrier or excipient.

35. The method according to claim 26 or claim 30, wherein the compound is administered via subcutaneous injection.

36. The method according to claim 26 or claim 30, wherein the compound is administered via inhalation.

37. A pharmaceutical composition for the sensitization of insulin in a mammal comprising a compound of the following formula: 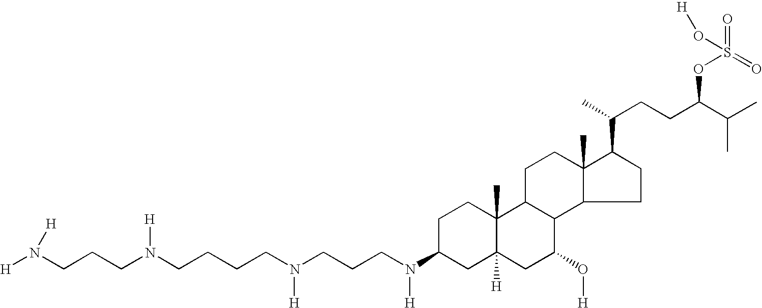
or a pharmaceutically acceptable salt thereof.
